(19) United States
(12) Patent Application Publication
(10) Pub. No.: US 2024/0144286 A1
(43) Pub. Date: May 2, 2024

(54) BITWISE RULES MATCHING ENGINE

(71) Applicant: Brightree LLC, Peachtree Corners, GA (US)

(72) Inventors: Jennifer A. KEISER VOAS, Cumberland, RI (US); Michael K. MILLER, Woodstock, GA (US); Howard Ashley PIERCE-MORGAN, Aberdeen (GB); Dean BENNETT, Peachtree Corners, GA (US); Sam MAKONNEN, Peachtree Corners, GA (US); Stuart HOOD, Paisley (GB); Kim M. BECKER, Lehigh Acres, FL (US); Graham Macgregor PATERSON, Bellshill (GB); Robert M. WRIGHT, Brevard, NC (US)

(21) Appl. No.: 18/495,487

(22) Filed: Oct. 26, 2023

Related U.S. Application Data
(60) Provisional application No. 63/420,931, filed on Oct. 31, 2022.

(51) Int. Cl. G06Q 20/40

(52) U.S. Cl. CPC G06Q 20/405

(57) ABSTRACT

Techniques for patient medical care are disclosed. These techniques include identifying a plurality of rules relating to a purchase of a medical item. The techniques further include selecting a first rule, of the plurality of rules, to apply to the purchase, including generating a specificity score for each of the plurality of rules, based on setting one or more bit values in each specificity score using matching criteria between the respective rule and the purchase and comparing the specificity scores for the plurality of rules. The techniques further include facilitating supply of the medical item to a patient based on completing the purchase using the selected first rule.

### CROSS-REFERENCE TO RELATED APPLICATIONS

This application claims priority to U.S. Provisional Patent Application No. 63/420,931, filed Oct. 31, 2022, the entire content of which is incorporated herein by reference in its entirety.

### INTRODUCTION

Aspects of the present disclosure relate to patient medical care, and more specifically, to improved supply for medical items, including pharmaceuticals.

Providing medical items to patients is very challenging. The pricing for medical items (e.g., pharmaceuticals, medical devices, and many other medical items) is typically governed by one or more contracts between a payer for the item (e.g., health insurer or care provider) and a seller of the item (e.g., another care provider, a manufacturer, or a distributer of the item). These contracts can include a very large number of rules governing various types and categories of items. Identifying applicable rules, selecting from among the applicable rules, and applying the selected rules is very challenging. Further, errors in this process can result in extra work for the seller in submitting claims to payers, extra work for the payer in adjudicating insurance claims, lost revenue on the part of the seller, as well as delays to patient care, resulting in less desirable outcomes.

### SUMMARY

Embodiments include a computer-implemented method. The method includes identifying a plurality of rules relating to a purchase of a medical item. The method further includes selecting a first rule, of the plurality of rules, to apply to the purchase, including: generating a specificity score for each of the plurality of rules, based on setting one or more bit values in each specificity score using matching criteria between the respective rule and the purchase, and comparing the specificity scores for the plurality of rules. The method further includes facilitating supply of the medical item to a patient based on completing the purchase using the selected first rule.

Embodiments further include an apparatus including a memory and a hardware processor communicatively coupled to the memory, the hardware processor configured to perform operations. The operations include identifying a plurality of rules relating to a purchase of a medical item. The operations further include selecting a first rule, of the plurality of rules, to apply to the purchase, including: generating a specificity score for each of the plurality of rules, based on setting one or more bit values in each specificity score using matching criteria between the respective rule and the purchase, and comparing the specificity scores for the plurality of rules. The operations further include facilitating supply of the medical item to a patient based on completing the purchase using the selected first rule.

Embodiments further include a non-transitory computer-readable medium including instructions that, when executed by a processor, cause the processor to perform operations. The operations include identifying a plurality of rules relating to a purchase of a medical item. The operations further include selecting a first rule, of the plurality of rules, to apply to the purchase, including: generating a specificity score for each of the plurality of rules, based on setting one or more bit values in each specificity score using matching criteria between the respective rule and the purchase, and comparing the specificity scores for the plurality of rules. The operations further include facilitating supply of the medical item to a patient based on completing the purchase using the selected first rule.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts a computing environment for a bitwise rules matching engine, according to one embodiment.

FIG. 2 depicts a block diagram for a controller for a bitwise rules matching engine, according to one embodiment.

FIG. 3 is a flowchart illustrating a bitwise rules matching engine, according to one embodiment.

FIG. 4 is a flowchart illustrating determining a best match rule for a bitwise rules matching engine, according to one embodiment.

FIG. 5 is a flowchart illustrating calculating rule match components for a bitwise rules matching engine, according to one embodiment.

FIGS. 6A-B illustrates bitwise matching for a bitwise rules matching engine, according to one embodiment.

FIG. 7 is a flowchart illustrating comparing specificity scores for a bitwise rules matching engine, according to one embodiment.

FIG. 8 illustrates a rules hierarchy for a bitwise rules matching engine, according to one embodiment.

FIG. 9 illustrates identifying applicable rules for a contract using a bitwise rules matching engine, according to one embodiment.

FIG. 10 is a flowchart illustrating identifying applicable rules for a contract using a bitwise rules matching engine, according to one embodiment.

FIG. 11 illustrates importing contract rules for a bitwise rules matching engine, according to one embodiment.

FIG. 12 is a flowchart illustrating importing contract rules for a bitwise rules matching engine, according to one embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved supply of medical items. As discussed above, determining pricing for medical items is very challenging. Existing systems require significant manual intervention and analysis, which results in risks to patient outcomes and many inefficiencies.

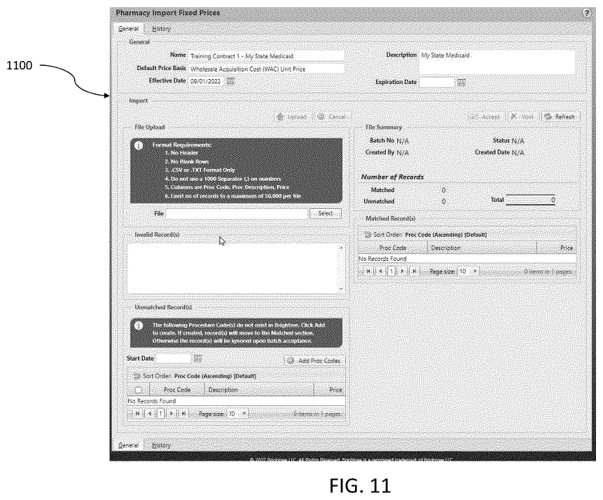

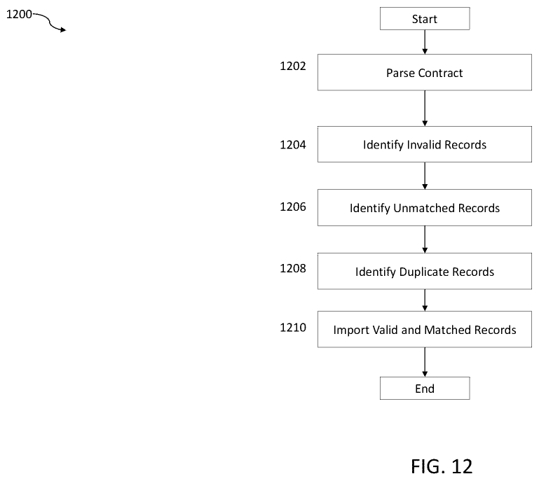

One or more techniques disclosed herein relate to bitwise rules matching that can be used to quickly, efficiently, and accurately identify appropriate pricing rules for purchase of a given item (e.g., a given medical item). In an embodiment, a contract (e.g., between a payer and a seller) includes numerous rules that govern pricing of medical items. These rules can be extracted from the contract (e.g., automatically as discussed further below with regard to FIGS. 11-12, or manually) and maintained in a rules repository.

Numerous rules may be applicable to a purchase of a given item. For example, a contract could include rules that apply to particular items, particular groups of items, particular Food and Drug Administration (FDA) categories of items (e.g., generic or brand pharmaceuticals), particular therapy types, particular cost ranges, and many other criteria. Identifying which of these rules should be applied to a given purchase is very challenging.

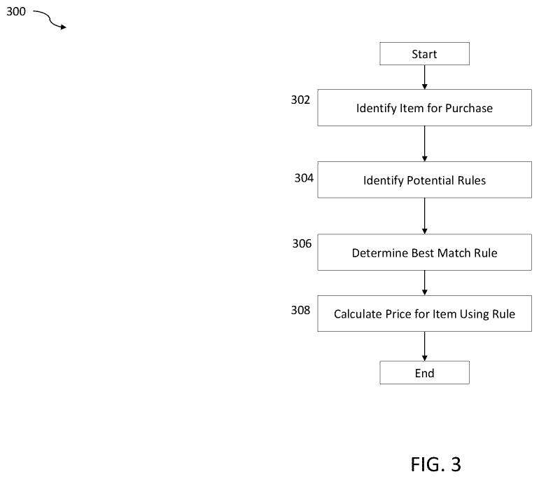

As discussed further below with regard to FIGS. 3-7, one or more techniques described herein address this problem by generating a bitwise specificity score for each potentially applicable rule. The specificity score for a given rule and purchase combination is generated by setting the value of bits in a variable representing the specificity score (e.g., a multiple-bit variable used in a computing system) based on which criteria in the rule apply to the purchase. The resulting specificity scores associated with various rules are then compared to determine which rule, or rules, are a best match for a given purchase and should be applied. For example, these bitwise specificity scores can be used to identify which rule, from among a number of potentially applicable rules, is most specifically applicable to the purchase and should be applied.

In an embodiment, one or more of these techniques provide technical improvements to existing medical supply systems. For example, pricing for items in existing systems is typically done manually (e.g., based on human review of applicable rules) or based on inefficient electronic comparison of rules. This results in both inaccurate pricing, and waste of human and computational resources. In particular, pricing of medical items is done extremely frequently and must provide quick results. Using the bitwise rules matching discussed below allows for rapid, and relatively computationally cheap, identification and application of pricing rules.

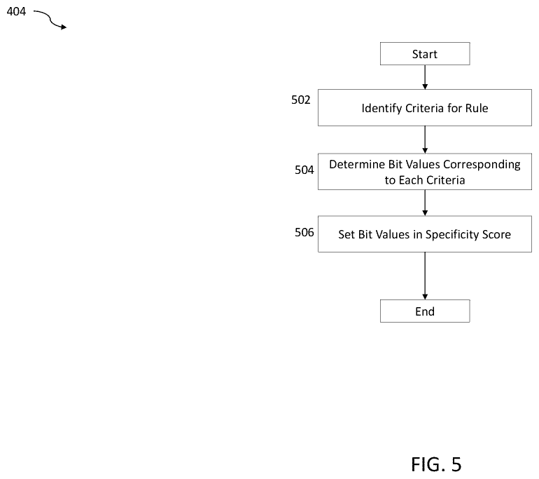

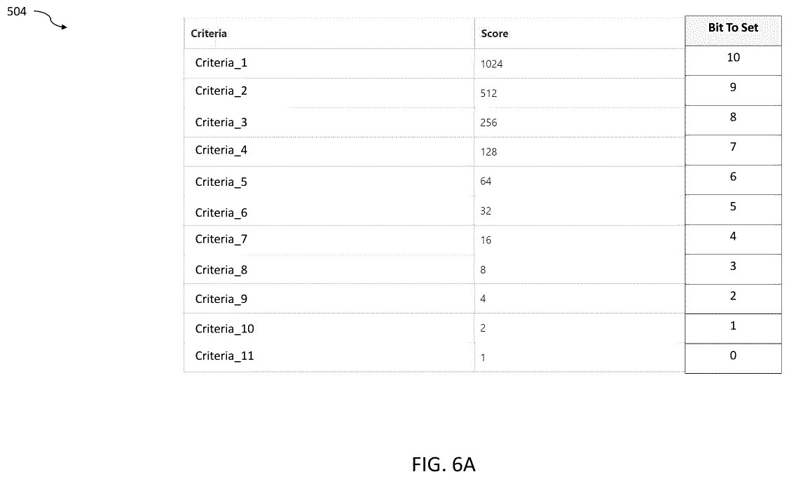

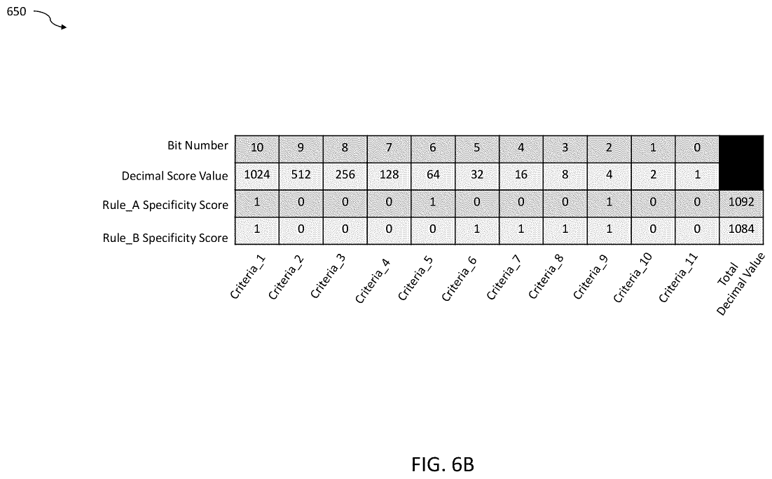

For example, assume a contract has dozens (or hundreds) of potentially applicable rules. Existing electronic systems do not include an efficient technique to compare these rules and select a best match rule for a given purchase. By using the bitwise specificity score discussed in relation to FIGS. 5 and 6A-B, below, one or more embodiments described herein allow for efficient and rapid rules matching. This is because computational devices store variables in memory in a binary representation made up of a number of bits. Setting the values of these bits based on matching criteria between a given rule and a given purchase can be done extremely quickly, and provides for highly computationally efficient comparisons. This improves the computational efficiency of the electronic systems and reduces computational resources used, as compared to prior techniques.

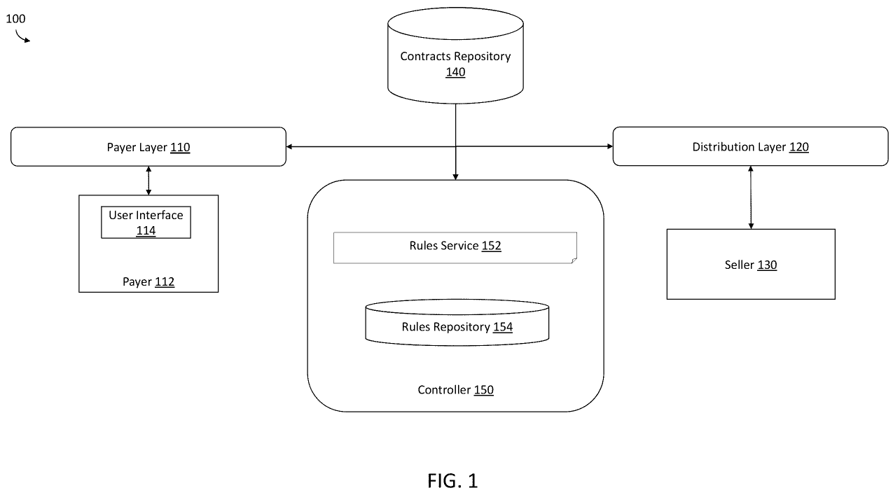

FIG. 1 depicts a computing environment 100 for a bitwise rules matching engine, according to one embodiment. In an embodiment, a payer 112 can interact with a payer layer 110 and a distribution layer 120 to purchase items (e.g., medical supplies) from a seller 130 (e.g., using a suitable electronic device). For example, the payer can be an insurer, a medical care provider, a patient, or any other person or entity purchasing items from the seller 130.

In an embodiment, the payer 112 can use any suitable electronic or computing device, including a smartphone, a tablet, a laptop computer, a desktop computer, a wearable device, a medical device, an Internet of Things (IoT) device, or any other suitable device. For example, the payer 112 can use a device that includes a user interface 114 to present information to the payer 112. The user interface 114 can present information to the payer 112 visually (e.g., using a display screen), through audio (e.g., using one or more speakers), or using any other suitable technique. Further, the user interface 114 can allow the payer 112 to interact with the electronic device (e.g., using a keyboard, mouse, touch sensitive interface, voice interface using one or more microphones, vision-tracking interface, or any other suitable interface).

In an embodiment, the price of items provided by the seller 130 is governed by one or more rules stemming from contracts maintained in a contracts repository 140. For example, the price of a particular medical item (e.g., a pharmaceutical or medical device) to a given payer 112 can be governed by a rule specified in a contract between the payer 112 and the seller 130, or another entity. In an embodiment, the payer 112 is a health insurer. Alternatively, the payer 112 is another healthcare entity (e.g., a care provider, a hospital, or another healthcare entity) or the patient themselves. The contracts in the contract repository 140 can specify a large number of rules potentially applicable to a given purchase of a medical item by the payer 112. In an embodiment, the contracts repository 140 is any suitable electronic storage repository, including an electronic database (e.g., a relational database, a graph database, or any other suitable database), a cloud storage location (e.g., a public cloud, a private cloud, or a hybrid cloud), an on-site or remote storage location, a distributed storage location (e.g., a blockchain), or any other suitable electronic storage repository.

In an embodiment, a controller 150 determines pricing for items purchased by the payer 112 from the seller 130. For example, the controller 150 can identify rules governing the pricing based on contracts maintained in the contracts repository 140. The controller 150 can maintain these rules in a rules repository 154. In an embodiment, the controller 150 can automatically parse the contracts from the contracts repository 140 to generate the rules in the rules repository 154. This is discussed further, below, with regard to FIGS. 11-12. Alternatively, or in addition, the rules can be added to the rules repository 154 manually (e.g., entered by a human based on a contract). In an embodiment, the rules repository 154 is any suitable electronic storage repository, including an electronic database (e.g., a relational database, a graph database, or any other suitable database), a cloud storage location (e.g., a public cloud, a private cloud, or a hybrid cloud), an on-site or remote storage location, a distributed storage location (e.g., a blockchain), or any other suitable electronic storage repository.

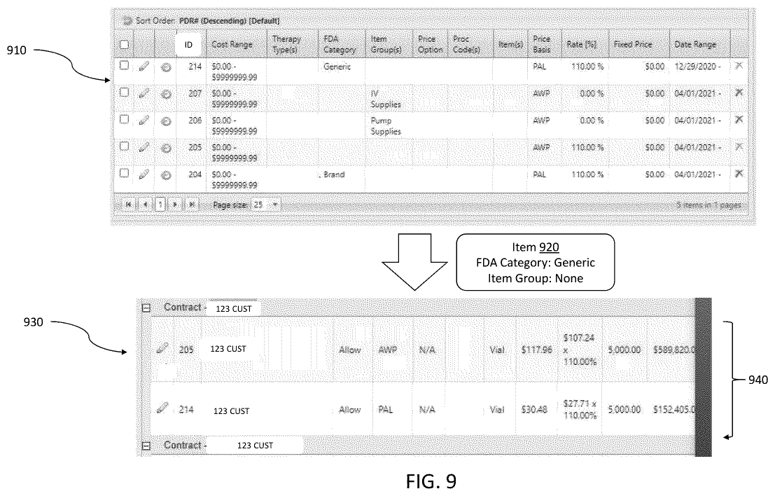

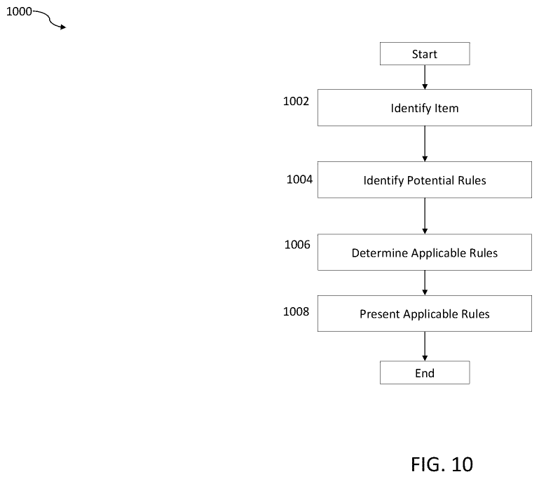

In an embodiment, the controller 150 includes a rules service 152. The rules service 152 facilitates identifying applicable rules for a given purchase, and determining pricing for the purchase based on the rules. For example, as discussed further below with regard to FIGS. 3-8, the rules service 152 can identify a suitable matching rule for a given purchase, from among a number of candidate rules, using a bitwise specificity score for each of the candidate rules. As another example, as discussed further below with regard to FIGS. 9-10, the rules service 152 can identify all applicable rules for a given contract in the contracts repository 140.

In an embodiment, the various components of the computing environment 100 communicate using one or more suitable communication networks, including the Internet, a wide area network, a local area network, or a cellular network, and uses any suitable wired or wireless communication technique (e.g., WiFi or cellular communication). Further, in an embodiment, the payer layer 110, controller 150, distribution layer 120, contracts repository 140, and seller 130, can be implemented using any suitable combination of physical compute systems, cloud compute nodes and storage locations, or any other suitable implementation. For example, the payer layer 110, controller 150, distribution layer 120, contracts repository 140, and seller 130, could each be implemented using a respective server or cluster of servers. As another example, the payer layer 110, controller 150, distribution layer 120, contracts repository 140 and seller 130, can be implemented using a combination of compute nodes and storage locations in a suitable cloud environment. For example, one or more of the components of the payer layer 110, controller 150, distribution layer 120, contracts repository 140, and seller 130, can be implemented using a public cloud, a private cloud, a hybrid cloud, or any other suitable implementation.

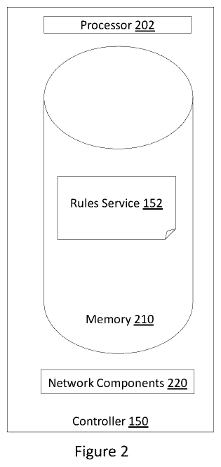

FIG. 2 depicts a block diagram for a controller 150 for a bitwise rules matching engine, according to one embodiment. The controller 150 includes a processor 202, a memory 210, and network components 220. The memory 210 may take the form of any non-transitory computer-readable medium. The processor 202 generally retrieves and executes programming instructions stored in the memory 210. The processor 202 is representative of a single central processing unit (CPU), multiple CPUs, a single CPU having multiple processing cores, graphics processing units (GPUs) having multiple execution paths, and the like.

The network components 220 include the components necessary for the controller 150 to interface with a suitable communication network (e.g., a communication network interconnecting various components of the computing environment 100 illustrated in FIG. 1, or interconnecting the computing environment 100 with other computing systems). For example, the network components 220 can include wired, WiFi, or cellular network interface components and associated software. Although the memory 210 is shown as a single entity, the memory 210 may include one or more memory devices having blocks of memory associated with physical addresses, such as random access memory (RAM), read only memory (ROM), flash memory, or other types of volatile and/or non-volatile memory.

The memory 210 generally includes program code for performing various functions related to use of the controller 150. The program code is generally described as various functional “applications” or “modules” within the memory 210, although alternate implementations may have different functions and/or combinations of functions. Within the memory 210, the rules service 152 facilitates identifying suitable rules for a given purchase (e.g., a purchase of a medical item by the payer 112 from the seller 130 illustrated in FIG. 1). This is discussed further, below, with regard to FIGS. 3-8. Alternatively, or in addition, the rules service 152 facilitates identifying applicable rules for a given contract (e.g., a contract maintained in the contracts repository 140 illustrated in FIG. 1). This is discussed further, below, with regard to FIGS. 9-10. Further, the rules service 152 can facilitate automatic generation of rules from a given contract (e.g., populating the rules repository 154 from one or more contracts in the contracts repository 140 illustrated in FIG. 1). This is discussed further, below, with regard to FIGS. 11-12.

While the controller 150 is illustrated as a single entity, in an embodiment, the various components can be implemented using any suitable combination of physical compute systems, cloud compute nodes and storage locations, or any other suitable implementation. For example, the controller 150 could be implemented using a server or cluster of servers. As another example, the controller 150 can be implemented using a combination of compute nodes and storage locations in a suitable cloud environment. For example, one or more of the components of the controller 150 can be implemented using a public cloud, a private cloud, a hybrid cloud, or any other suitable implementation.

Although FIG. 2 depicts the rules service 152 as being located in the memory 210, that representation is also merely provided as an illustration for clarity. More generally, the controller 150 may include one or more computing platforms, such as computer servers for example, which may be co-located, or may form an interactively linked but distributed system, such as a cloud-based system, for instance. As a result, the processor 202, and the memory 210, may correspond to distributed processor and memory resources within the computing environment 100. Thus, it is to be understood that the rules service 152 may be stored at any suitable location within the distributed memory resources of the computing environment 100.

FIG. 3 is a flowchart 300 illustrating a bitwise rules matching engine, according to one embodiment. At block 302 a rules service (e.g., the rules service 152 illustrated in FIGS. 1-2) identifies an item for purchase. For example, a payer (e.g., the payer 112 illustrated in FIG. 1) can seek to purchase a medical item (e.g., a pharmaceutical or medical device, for use by the payer or by a patient affiliated with the payer). The rules service can identify the item sought for purchase. While FIG. 3 focuses on purchase of a medical item, this is merely one example. In an embodiment, the techniques discussed with regard to FIG. 3 (and subsequent figures) can be applied to purchase of any item in which the price is governed by a number of potentially applicable rules.

At block 304, the rules service identifies potential rules. In an embodiment, the rules service identifies potentially applicable rules in a rules repository (e.g., the rules repository 154 illustrated in FIG. 1). For example, a contract between the payer and a seller of the desired item (e.g., the seller 130 illustrated in FIG. 1) can govern prices for the item. This contract can be stored in a contract repository (e.g., the contracts repository 140 illustrated in FIG. 1) and can be used to populate the rules repository. The rules service can retrieve potentially applicable rules from the rules repository (e.g., using a database query, by retrieving from a cloud storage location or other suitable storage location, or using any other suitable technique). In an embodiment, the potentially applicable rules include all rules defined in the contract between the payer and the seller. Alternatively, the rules service can filter the rules defined in the contract to exclude inapplicable rules.

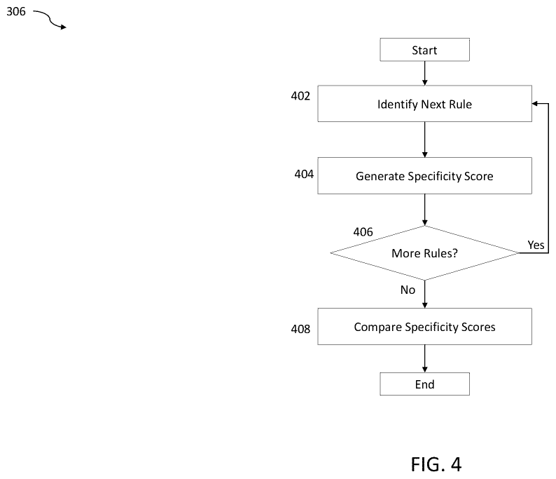

At block 306, the rules service determines a best match rule for the purchase. For example, the rules service can identify which of the rules identified at block 304 should be applied to the purchase. This can be done by using a bitwise rules comparison to rapidly, and accurately, identify which of the potentially applicable rules are the best match. This is discussed further, below, with regard to FIG. 4.

FIG. 4 focuses on identifying a single best match for a rule and an item purchase. Alternatively, or in addition, in an embodiment the rules service can identify (and apply) multiple rules. For example, multiple rules may be suitable for a given purchase. The rules service can identify which rules should be used, and can apply all the selected rules, or a subset of the selected rules.

At block 308, the rules service calculates a price for the item using the rule (e.g., the best match rule identified at block 306). In an embodiment, a rule can govern the price in a variety of different ways. For example, the rule can specify a fixed price for the item. As another example, the rule can specify a discounted price for the item (e.g., a discount off a published price price). In an embodiment, the rules service can retrieve published prices (e.g., from a suitable published listing) and can use the published prices to calculate the prices. As another example, the rule can specify a formula to use in calculating the price for the item (e.g., based on the published price or another value). These are all merely examples, and the rule can govern the price using any suitable technique. In an embodiment, the calculated price can be used to purchase the item and facilitate supply of the item to the patient.

FIG. 4 is a flowchart illustrating determining a best match rule for a bitwise rules matching engine, according to one embodiment. In an embodiment, FIG. 4 corresponds with block 306 illustrated in FIG. 3. At block 402, a rules service (e.g., the rules service 152 illustrated in FIGS. 1-2) identifies a next rule. For example, the rules service can iterate through a list of potentially applicable rules (e.g., identified at block 304 discussed above in relation to FIG. 3), and can select the next potentially applicable rule.

At block 404, the rules service generates a specificity score for the identified rule. In an embodiment, the rules service can identify one or more criteria for the rule, determine bit values corresponding to each of the criteria, and generate a specificity score by setting the relevant bit values in in the specificity score. For example, a given rule may apply to a particular item, a particular group of items, a particular medical procedure identified by a procedure code, a particular FDA category, a particular therapy type, a particular amount, or any other suitable criteria. These are merely examples. As illustrated further in FIGS. 6A-B, each criterion can be associated with a particular bit value, and the rules service can determine the bit values corresponding to the identified criterion. The rules service can then set bit values in a specificity score variable based on the determined bit values (e.g., bits corresponding to criteria applicable to the rule are set to high (“1”) while bits corresponding to criteria not applicable to the rule are set to low (“0”)). This is discussed further, below, with regard to FIGS. 5 and 6A-B.

At block 406, the rules service determines whether there are more rules. For example, the rules service can determine whether there are more potentially applicable rules for which a specificity score should be generated. If so, the flow returns to block 402 and the rules service identifies the next rule. If not, the flow proceeds to block 408.

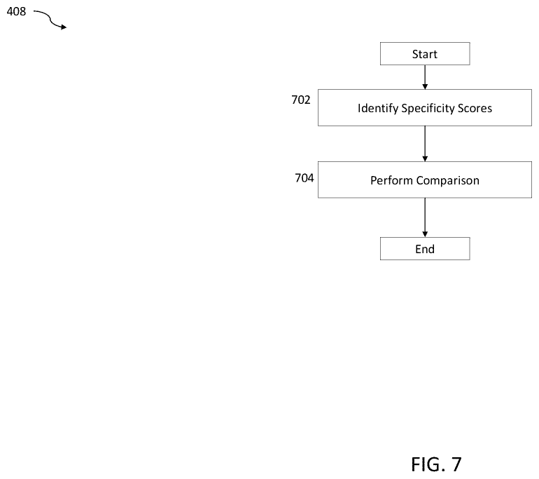

At block 408, the rules service compares specificity scores. For example, the rules service can identify the specificity score variables generated for each potentially applicable rule (e.g., generated at block 404 discussed above). The rules service can then perform a comparison of the specificity scores, to identify the best match rule. This is discussed further, below, with regard to FIG. 7.

FIG. 5 is a flowchart illustrating calculating rule match components for a bitwise rules matching engine, according to one embodiment. In an embodiment, FIG. 5 corresponds with block 404 illustrated in FIG. 4. At block 502, a rules service (e.g., the rules service 152 illustrated in FIGS. 1-2) identifies one or more criteria for a rule. For example, as discussed above, a given rule may apply to a particular item, a particular group of items, a particular medical procedure identified by a procedure code, a particular Food and Drug Administration (FDA) category, a particular therapy type, a particular amount, or any other suitable criteria. The rules service can identify all criteria applicable to a rule (e.g., based on characteristics, properties, or meta-data associated with the rule).

At block 504, the rules service determines bit values corresponding to each criteria. In an embodiment, each criteria is tied to a specific bit in a specificity score variable. FIGS. 6A-B provide an example by illustrating bitwise matching for a bitwise rules matching engine, according to one embodiment. FIG. 6A illustrates a number of criteria and a corresponding bit, for each criterion, defined on a bitwise basis from a most significant bit to a least significant bit.

Assume the specificity score is a variable made up of at least 11 bits. Each bit, from bit 0 (e.g., the least significant bit) to bit 10 (e.g., the most significant bit of the 11 relevant bits) is associated with a criterion. If the rule matches a purchase for a given criterion then the corresponding bit is set to high (e.g., “1”). If the rule does not match a purchase for a given criterion, then the corresponding bit is set to low (e.g., “0”). In an embodiment, the criteria are ordered from most specific (Criteria_1) to least specific (Criteria_11). For example, Criteria_1 may indicate that the relevant rule relates to the specific item being purchased, while Criteria_11 may indicate that the relevant rule relates to the specified amount of the item (e.g., a 30 day supply, a 7 day supply, a specified quantity of the item, or any other suitable amount).

In an embodiment, as discussed above, the rule that most specifically matches the item being purchased should be used. For example, assume two potential rules are available: a rule relating to the specific item being purchased, and a rule relating to any item but relating to the desired quantity for purchase. The rule relating to the item matches the purchase more specifically than the rule relating to the desired quantity, and so the rule relating to the specific item should be used. Setting the bit values as illustrated in FIG. 6A allows the rules service to accurately, and efficiently, identify the most specific rule applicable to a desired purchase.

As illustrated in FIG. 6A, Criteria_2 is associated with bit 9, while Criteria_6 is associated with bit 5. Assume Criteria_2 reflects that the rule is associated with a particular group of items, while Criteria_6 reflects that the rule is associated with a particular therapy type. A rule that matches the item for purchase in both criteria (i.e., the rule relates both to a group of items including the item for purchase and a therapy for which the item is being used) would have both bit 9 and bit 5 set to high. A rule that matches the item for purchase in neither criteria would have both bit 9 and bit 5 set to low. A rule that matches the item for purchase in category of items, but not therapy type, would have bit 9 set to high and bit 5 set to low.

Returning to FIG. 5, at block 506 the rules service sets the bit values in the specificity score. For example, as discussed above, each criterion is associated with a particular bit in a specificity score variable. The rules service sets the corresponding bit, for each matching criterion.

For example, as illustrated in a table 650 illustrated in FIG. 6B, assume a specificity score includes at least 11 bits. Each criterion is associated with a designated one of those bits. Assume a Rule_A corresponds with an item for purchase for Criteria_1, Criteria_5, and Criteria_9. The rules service sets the corresponding bits 10, 6, and 2 to high. Assume a Rule_B corresponds with an item for purchase at different criteria: Criteria_1, Criteria_6, Criteria_7, Criteria_8, and Criteria_9. The rules service sets the corresponding bits 10, 5, 4, 3, and 2 to high.

In an embodiment, setting the specificity score in a bitwise fashion guarantees that any given combination of criteria will result in a unique specificity score. For example, a rule matching with the desired purchase on any set of criteria will be guaranteed to produce a different specificity score from a different rule matching with the desired purchase on any different set of criteria. This ensures that when comparing specificity scores, as discussed further below with regard to FIG. 7, two rules will only have the same specificity score if they match the desired purchase on exactly the same criteria (e.g., there will be no ties for rules matching on different criteria).

FIG. 7 is a flowchart illustrating comparing specificity scores for a bitwise rules matching engine, according to one embodiment. In an embodiment, FIG. 7 corresponds with block 408 illustrated in FIG. 4. At block 702 a rules service (e.g., the rules service 152 illustrated in FIGS. 1-2) identifies specificity scores. For example, the rules service can identify all specific scores generated at block 404, illustrated in FIG. 4.

At block 704, the rules service performs the comparison. In an embodiment, the rules service compares the values of the specificity scores and selects the highest value as the best match. For example, the rules service can use binary arithmetic to compare the bit values of the specificity scores and select the highest value. As another example, the rules service can compare decimal values of the specificity scores.

In an embodiment, both scenarios are illustrated in FIG. 6B. For example, the rules service can compare the values of the Rule_A specificity score and the Rule_B specificity score. In an embodiment, the rules service performs a binary comparison and identifies the Rule_A specificity score as having a higher value. Alternatively, the rules service identifies the decimal values of the specificity scores and determines that the Rule_A specificity score has a higher total decimal value than the Rule_B specificity score.

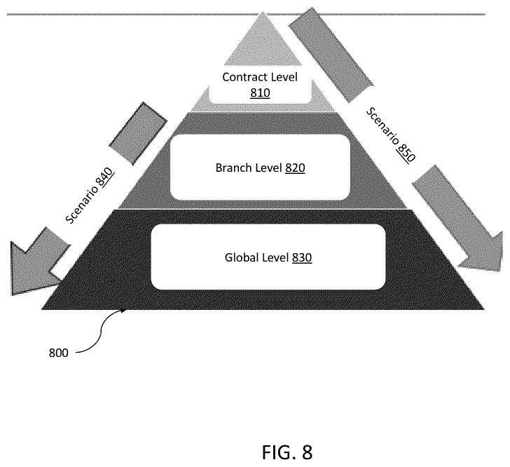

FIG. 8 illustrates a rules hierarchy for a bitwise rules matching engine, according to one embodiment. In an embodiment, the rules used to govern prices for a healthcare organization are ordered hierarchically. For example, a rules hierarchy 800 includes three hierarchical levels: a contract level 810, which sets prices that are specific to one or more payers; a branch level 820, which sets prices for any payer billed by the healthcare organization's branch location; and a global level 830, which is used when there is no matching price rules defined at any other level of the hierarchy. In an embodiment, a rules service (e.g., the rules service 152 illustrated in FIGS. 1-2) identifies a best match rule (e.g., at block 306 illustrated in FIG. 3) in a hierarchal fashion based on both the specificity score and the hierarchical level associated with the rule.

For example, each of the hierarchical levels 810, 820, and 830 can include a collection of rules. In an embodiment, the rules service can identify potential rules, generate specificity scores, and determine a best match rule based on the specificity scores (e.g., as discussed above in relation to FIGS. 3-7) for multiple levels in the rules hierarchy. For example, in a scenario 850 the rules service can identify potential rules, generate specificity scores, and determine a best match rule based on the specificity scores for each of the levels 810, 820, and 830. As another example, in a scenario 840 the rules service can identify potential rules, generate specificity scores, and determine a best match rule based on the specificity scores for only the lower two levels 820 and 830. These are merely example, and any number of levels and combination of levels can be used.

The rules service can then select a best match rule based on both the specificity score and the level in the rules hierarchy. For example, a rule relating to the contract level 810 might be preferred over a rule relating to the global level 830. The rules service can select the best match rule based on both this hierarchical relationship and the specificity score.

FIG. 9 illustrates identifying applicable rules for a contract using a bitwise rules matching engine, according to one embodiment. As discussed above, in one embodiment a rules service (e.g., the rules service 152 illustrated in FIGS. 1-2) identifies a best match rule for a given purchase. Alternatively, or in addition, the rules service identifies and presents to a user all potentially applicable rules for an item from a contract.

For example, a contract 910 includes five rules, with identifiers 204, 205, 206, 207, and 214. Rule 214 applies only to items in the FDA category “Generic,” while Rule 204 applies only to items in the FDA category “Brand.” Rules 205, 206, and 207 do not specify an FDA category, and so apply to all FDA categories. Further, Rule 207 applies only to items in the Item Group “IV Supplies” while Rule 206 applies only to items in the Item Group “Pump Supplies.” Rules 204, 205, and 214 do not specify an Item Group and so apply to all item Groups.

Assume a user selects an item 920 for analysis. The item 920 is in the FDA category “Generic” and does not have an associated Item Group. In an embodiment, the rules service generates a user interface 930 presenting the potentially applicable rules from the contract 910 for the item 920. The item 920 is in the FDA category “Generic,” so Rule 204 is excluded. The item 920 does not have an associated Item Group, so rules 206 and 207 are excluded. Thus, the rules service presents rules 205 and 214 as potential rules from the contract 910 for the item 920. Further, the rules service presents an estimated item cost 940 for the item 920 based on each of the rules 205 and 214. In an embodiment, as discussed below in relation to FIG. 10, the bitwise rules matching techniques discussed above in relation to FIGS. 3-8 are used to identify the potential rules.

FIG. 10 is a flowchart 1000 illustrating identifying applicable rules for a contract using a bitwise rules matching engine, according to one embodiment. At block 1002 a rules service (e.g., the rules service 152 illustrated in FIGS. 1-2) identifies an item. For example, the rules service can identify the item for purchase (e.g., the item 920 illustrated in FIG. 9).

At block 1004, the rules service identifies all potential rules for the item from a given contract. For example, returning to FIG. 9, the rules service could identify all of rules 204, 205, 206, 207, and 214, from the contract 910, as potentially applicable rules.

At block 1006, the rules service determines applicable rules. For example, returning to FIG. 9, the rules service could identify rules 205 and 214 as potentially applicable rules. In an embodiment, the rules service identifies applicable criteria for each rule, and the item for purchase, and generates a specificity score for each rule. This is discussed further above in relation to FIGS. 5 and 6A-B. The rules service can then use this specificity score to determine potentially applicable rules. For example, a rule with a specificity score greater than zero may be considered potentially applicable. Alternatively, or in addition, a rule with a specificity score of zero may be considered potentially applicable to all items. These are merely examples, and any suitable technique can be used.

At block 1008, the rules service presents the applicable rules. For example, as illustrated in FIG. 9, the rules service can generate a user interface (e.g., the user interface 930) to present the applicable rules to the user.

FIG. 11 illustrates importing contract rules for a bitwise rules matching engine, according to one embodiment. As discussed above in relation to FIG. 1, in an embodiment a controller 150 uses a rules repository 154 including rules generated from contracts stored in a contracts repository 140. In one embodiment, these rules are manually added to the rules repository 154 (e.g., by a human administrator reviewing the contract and entering the rules). Alternatively, a rules service (e.g., the rules service 152 illustrated in FIGS. 1-2) automatically parses a contract and generates the rules.

For example, a user interface 1100 allows a user to upload a file relating to a contract, and generates a number of rules from the contract. For example, the user can upload the contract in comma separated (e.g., CSV) or textual form. The rules service can then parse the contract to identify rules included in the contract. Successfully parsed rules are considered Matched Records, and are added to a rules repository.

In an embodiment, however, the rules service can identify two categories of unsuccessfully parsed records: invalid records, and unmatched record. In an embodiment, invalid records do not match an expected format. For example, a user may have imported a file that does not include rules in an expected format, or that does not include rules at all. The rules service identifies these as invalid records, and presents them to the user for review.

In an embodiment, unmatched records are formatted correctly but are not associated with known identifiers (e.g., known procedure codes). For example, rules must be correlated with a suitable identifier (e.g., a procedure code) to be used in the system. A rule that does not match a known procedure code is considered an unmatched record, and presented to the user. The user can then add the procedure code (e.g., so that the record is no longer unmatched), correct an error in the procedure code imported from the contract, or take any other suitable action to correct the problem. In an embodiment, procedure codes added by the user are recorded for that entity, and can be used for future rules imports.

FIG. 12 is a flowchart 1200 illustrating importing contract rules for a bitwise rules matching engine, according to one embodiment. In an embodiment, at block 1202 a rules service (e.g., the rules service 152 illustrated in FIGS. 1-2) parses a contract. For example, as illustrated in FIG. 11, a user can select a file to upload and import a contract. The rules service can parse the contract. In an embodiment, the rules service parses the contract based on an expected format for rules in the contract. Alternatively, or in addition, the rules service can use natural language processing (NLP) to parse the contract. For example, the rules service can use a suitable machine learning (ML) model (e.g., a suitable trained ML model) to parse the contract and identify rules in the contract. This can allow the rules service to parse contracts in a wide variety of formats and including a wide variety of rules.

At block 1204, the rules service identifies invalid records. As discussed above, in an embodiment the rules service identifies rules in the contract that do not appear to be formatted correctly. The rules service can skip these rules, and present them to the user for correction (e.g., as illustrated in FIG. 9).

At block 1206, the rules service identifies unmatched records. As discussed above, in an embodiment each rule must be associated with a suitable identifier (e.g., a procedure code). The rules service can determine that a given rule is not associated with a known identifier, and can skip these rules and present them to the user for correction (e.g., as illustrated in FIG. 9). For example, the rules service can provide a user interface through which a user can add a missing identifier for a given entity (e.g., the entity for which these rules apply), or correct an identifier imported from a contract.

At block 1208, the rules service identifies duplicate records. In an embodiment, the rules service identifies rules in the contract that are duplicative. For example, multiple rules could match identically (e.g., in every aspect), or match across a number of criteria (e.g., match across a threshold number of criteria or across designated criteria). In an embodiment, the rules service can exclude duplicate records, present duplicate records for review and exclusion or approval by a user (e.g. using a suitable user interface), or can use any other suitable technique.

At block 1210, the rules service imports valid records. As discussed above, the rules service identifies rules that are both valid and matched, and imports these rules. The rules service further presents these rules to the user for review, in a suitable user interface.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in FIGS., those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A computer-implemented method, comprising:
identifying a plurality of rules relating to a purchase of a medical item;
selecting a first rule, of the plurality of rules, to apply to the purchase, comprising:
generating a specificity score for each of the plurality of rules, based on setting one or more bit values in each specificity score using matching criteria between the respective rule and the purchase;
comparing the specificity scores for the plurality of rules; and
facilitating supply of the medical item to a patient based on completing the purchase using the selected first rule.

2. The computer-implemented method of claim 1, wherein setting the one or more bit values in each specificity score using matching criteria between the respective rule and the purchase further comprises:
identifying a plurality of criteria relating to the plurality of rules;
determining the matching criteria from among the plurality of criteria;
determining a bit value relating to each of the matching criteria; and
setting the one or more bit values in each specificity score based on the determined bit values relating to the matching criteria.

3. The computer-implemented method of claim 2, wherein the bit value relating to each of the matching criteria is defined on a bitwise basis from a most significant bit to a least significant bit.

4. The computer-implemented method of claim 2, wherein comparing the specificity scores for the plurality of rules comprises:
selecting a most specific of the plurality of rules based on the values of the specificity scores.

5. The computer-implemented method of claim 1, further comprising:
generating a plurality of specificity scores, relating to a plurality of rules, for each of a plurality of hierarchical levels.

6. The computer-implemented method of claim 1, further comprising:
identifying a second plurality of rules relating to purchase of medical items;
selecting one or more rules, from the second plurality of rules, that are potentially applicable to purchase of a second medical item, comprising:
generating a second plurality of specificity scores relating to the second plurality of rules; and
generating a user interface for presenting the selected one or more rules to a user.

7. The computer-implemented method of claim 6, wherein generating the second plurality of specificity scores relating to the second plurality of rules comprises setting one or more bit values for each of the second plurality of specificity scores based on matching criteria between the second plurality of rules and the second medical item.

8. The computer-implemented method of claim 1, further comprising:
generating the plurality of rules based on a contract relating to a payer for the purchase of the medical item.

9. The computer-implemented method of claim 8, wherein generating the plurality of rules based on the contract further comprises:
parsing a data structure relating to the contract to generate the plurality of rules; and
storing the generated plurality of rules in an electronic repository.

10. The computer-implemented method of claim 9, wherein parsing the data structure relating to the contract to generate the plurality of rules further comprises:
identifying one or more invalid records in the data structure;
identifying one or more unmatched records in the data structure; and
generating a user interface to present to the user the identified one or more invalid records and the identified one or more unmatched records.

11. An apparatus comprising:
a memory; and
a hardware processor communicatively coupled to the memory, the hardware processor configured to perform operations comprising:
identifying a plurality of rules relating to a purchase of a medical item;
selecting a first rule, of the plurality of rules, to apply to the purchase, comprising:
generating a specificity score for each of the plurality of rules, based on setting one or more bit values in each specificity score using matching criteria between the respective rule and the purchase;
comparing the specificity scores for the plurality of rules; and
facilitating supply of the medical item to a patient based on completing the purchase using the selected first rule.

12. The apparatus of claim 11, wherein setting the one or more bit values in each specificity score using matching criteria between the respective rule and the purchase further comprises:
identifying a plurality of criteria relating to the plurality of rules;
determining the matching criteria from among the plurality of criteria;
determining a bit value relating to each of the matching criteria; and
setting the one or more bit values in each specificity score based on the determined bit values relating to the matching criteria.

13. The apparatus of claim 12, wherein the bit value relating to each of the matching criteria is defined on a bitwise basis from a most significant bit to a least significant bit.

14. The apparatus of claim 12, wherein comparing the specificity scores for the plurality of rules comprises:
selecting a most specific of the plurality of rules based on the values of the specificity scores.

15. The apparatus of claim 11, further comprising:
generating a plurality of specificity scores, relating to a plurality of rules, for each of a plurality of hierarchical levels.

16. A non-transitory computer-readable medium comprising instructions that, when executed by a processor, cause the processor to perform operations comprising:
identifying a plurality of rules relating to a purchase of a medical item;
selecting a first rule, of the plurality of rules, to apply to the purchase, comprising:
generating a specificity score for each of the plurality of rules, based on setting one or more bit values in each specificity score using matching criteria between the respective rule and the purchase;
comparing the specificity scores for the plurality of rules; and
facilitating supply of the medical item to a patient based on completing the purchase using the selected first rule.

17. The non-transitory computer-readable medium of claim 16, wherein setting the one or more bit values in each specificity score using matching criteria between the respective rule and the purchase further comprises:
identifying a plurality of criteria relating to the plurality of rules;
determining the matching criteria from among the plurality of criteria;
determining a bit value relating to each of the matching criteria; and
setting the one or more bit values in each specificity score based on the determined bit values relating to the matching criteria.

18. The non-transitory computer-readable medium of claim 17, wherein the bit value relating to each of the matching criteria is defined on a bitwise basis from a most significant bit to a least significant bit.

19. The non-transitory computer-readable medium of claim 17, wherein comparing the specificity scores for the plurality of rules comprises:
selecting a most specific of the plurality of rules based on the values of the specificity scores.

20. The non-transitory computer-readable medium of claim 16, further comprising:
generating a plurality of specificity scores, relating to a plurality of rules, for each of a plurality of hierarchical levels.
